(19) United States
(12) Patent Application Publication
(10) Pub. No.: US 2025/0332327 A1
(43) Pub. Date: Oct. 30, 2025

(54) INTRALUMINAL PRESSURE PREDICTOR FOR FLUID MANAGEMENT SYSTEM

(71) Applicant: Boston Scientific Scimed Inc., Maple Grove, MN (US)

(72) Inventors: Jessica Williams, Marlborough, MA (US); Randall Maas, Chaska, MN (US); John O'Donnell, Tipperary (IE); Cassandra Anne Hailson, Marlborough, MA (US)

(73) Assignee: Boston Scientific Scimed Inc., Maple Grove, MN (US)

(21) Appl. No.: 19/188,070

(22) Filed: Apr. 24, 2025

Related U.S. Application Data
(60) Provisional application No. 63/640,377, filed on Apr. 30, 2024.

(51) Int. Cl. A61M 1/00; A61B 1/00; G16H 40/63

(52) U.S. Cl. CPC A61M 1/00; A61B 1/00119; G16H 40/63; A61B 1/00097; A61M 2202/0007; A61M 2202/04; A61M 2205/10; A61M 2205/12; A61M 2205/3327; A61M 2205/3331; A61M 2205/3365; A61M 2205/50

(57) ABSTRACT

The disclosure provides devices, systems, and methods to predict an intraluminal pressure (ILP) of a lumen, at a future time, in which an endoscopic medical device is inserted and coupled to a fluid management system (FMS) to provide fluid flow to the lumen. The disclosure provides a controller for the FMS to predict ILP of the lumen and to modulate operating parameters (e.g., speed, on/off, parasitic timing, flow direction, etc.) of a pump of the FMS based on the predicted ILP and an ILP threshold value to prevent or mitigate the risk that the ILP will exceed the ILP threshold value.

### CROSS-REFERENCE TO RELATED APPLICATIONS

This application claims the benefit of U.S. Provisional Application No. 63/640,377, filed Apr. 30, 2024, the disclosure of which is incorporated herein by reference in its entirety.

### TECHNICAL FIELD

The disclosure generally relates to a fluid management system and particularly, but not exclusively, to a system and method for stopping a pump in the fluid management system in a manner and time to mitigate overshooting an intraluminal pressure threshold.

### BACKGROUND

Flexible ureteroscopy (fURS), gynecology, and other endoscopic procedures require the circulation of fluid for several reasons. Fluid management systems may be used to deliver fluid to an anatomical cite from a reservoir at a desired pressure and/or flow rate via a peristaltic or roller pump. The fluid management system may utilize a fluid tubing set installed with a pump console to provide the fluid to the patient, often via a procedural device. Further, the fluid management system may adjust the flow rate and/or pressure at which fluid is delivered from the reservoir based on data collected from the procedural device, such as, but not limited to, pressure readings. There is an ongoing need to provide alternative configurations of the components of fluid management systems, to facilitate the use thereof.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

As noted, a fluid management system (FMS) can be utilized to deliver fluid to a procedure site (e.g., the urinary system, or the like) during a ureteroscopy procedure. When used in conjunction with certain procedural devices (e.g., LithoVue™ Elite, or the like), the FMS can measure the intraluminal pressure (ILP) and stop the pump if the ILP exceeds a chosen threshold. However, after disabling the pump, due for example, to the length of the fluid tubing set, fluid may continue to flow from the FMS to the procedure site, which can cause the ILP to continue to increase, even after the pump has stopped.

Further, the characteristics of the fluid outflow from the procedure site (e.g., a kidney, or the like) can cause ILP to continue to rise even after the pump has stopped. In either or both these examples, the ILP may increase past, and exceed, the threshold despite stopping the pump.

As another example, due to the capacity of the FMS to store fluid, the effects of the FMS pump speed (either increase or decrease) on ILP will not be measurable by sensors in the lumen for some amount of time (e.g., several seconds, or the like).

Additionally, an often-used feature of FMS is to aid clearing the visual field with fluid flow. To make this a responsive feature, for example, usable by a physician in real-time or on-demand, the pump of the FMS is often operated at a highly elevated speed (referred to as “boost”) when this feature is requested.

As a result of the above-described continued flow of fluid after disabling the pump, fluid dynamics related to FMS fluid supply, measurement delay, and operation of the FMS pump at boost speeds, techniques are needed to determine how to operate the pump and under what parameters such that ILP does not exceed a specified threshold. The present disclosure is directed towards identifying parameters for the pump of an FMS system that will mitigate or reduce the risk that the ILP (e.g., as measured by a procedural device) will exceed a threshold ILP value.

In some embodiments, the disclosure can be implemented as a method for a controller of a fluid management system (FMS) where the FMS is configured to provide fluid flow to an endoscopic medical device. The method can comprise receiving, at circuitry of the controller, an indication to operate a pump of the FMS at a specified parameter; determining, by the circuitry, a predicted intraluminal pressure (ILP) of a lumen at a time step in the future, wherein the endoscopic medical device comprises an elongated shaft inserted into the lumen and is configured to provide fluid from the FMS to the lumen; determining, by the circuitry, whether the predicted ILP is less than or equal to a threshold ILP level; and sending, responsive to a determination that the predicted ILP is not less than or equal to the threshold ILP level, a first control signal to the pump from the circuitry, the first control signal to cause the pump to operate at a parameter different than the specified parameter; or sending, responsive to a determination that the predicted ILP is less than or equal to the threshold ILP level, a second control signal to the pump from the circuitry, the second control signal to cause the pump to operate at the specified parameter.

With some embodiments of the method, the specified parameter comprises a desired RPM and the first control signal is configured to cause the pump to operate at an RPM different than the desired RPM.

With some embodiments of the method, the first control signal is configured to cause the pump to stop or to reverse flow direction.

With some embodiments, the method further comprises receiving, at the circuitry, an indication to operate the pump at a specified RPM, the first control signal is configured to cause the pump to operate at an RPM less than the specified RPM, and the second control signal is configured to cause the pump to operate at the specified RPM.

With some embodiments, the method further comprises receiving, at the circuitry from the pump, an indication of a current RPM of the pump; and determining, by the circuitry, the predicted ILP of the lumen at the time step in the future based in part on the current RPM of the pump and the specified RPM.

With some embodiments, the method further comprises receiving, at the circuitry from a medical device pressure sensor disposed in a distal end of the elongate shaft, an indication of a current ILP of the lumen; and determining, by the circuitry, the predicted ILP of the lumen at the time step in the future based in part on the current ILP of the lumen, the current RPM of the pump, and the specified RPM.

With some embodiments of the method, the FMS comprises a console configured to receive a fluid cassette, the pump, and an FMS pressure sensor configured to measure a pressure in the fluid cassette, and the pump is configured to cause fluid to flow through the fluid cassette, the method further comprising receiving, at the circuitry from the FMS pressure sensor, an indication of a current pressure of the fluid cassette; and determining, by the circuitry, the predicted ILP of the lumen at the time step in the future based in part on the current pressure of the fluid cassette, the current ILP of the lumen, the current RPM of the pump, and the specified RPM.

With some embodiments of the method, the fluid cassette comprises a housing defining a fluid pathway therethrough, the fluid pathway comprises a fluid dampening chamber having a fluid inlet configured for fluid ingress into the fluid dampening chamber and a fluid outlet configured for fluid egress from the fluid dampening chamber, the method further comprising determining, by the circuitry, the predicted ILP of the lumen at the time step in the future based in part on fluid dynamics of the fluid dampening chamber.

With some embodiments, the method further comprises solving, by the circuitry, a series of time-dependent differential equations; and determining, by the circuitry, the predicted ILP of the lumen at the time step in the future based on the solution to the series of time-dependent differential equations.

With some embodiments, the method further comprises solving, by the circuitry, the series of time-dependent differential equations using a forward-Euler algorithm or a backward-Euler algorithm.

With some embodiments of the method, the series of time-dependent differential equations relates a volume of the fluid cassette with the current ILP.

With some embodiments of the method, the series of time-dependent differential equations relates fluid outflow resistance of the lumen to fluid inflow resistance of the lumen.

With some embodiments of the method, the series of time-dependent differential equations is based in part on compliance of the lumen.

In some embodiments, the disclosure can be implemented as an apparatus to control a fluid management system (FMS) configured to provide fluid flow to an endoscopic medical device. The apparatus can comprise a processor coupled to a memory comprising instructions executable by the processor. The instructions when executed by the processor cause the apparatus to implement any of the method described herein.

In some embodiments, the disclosure can be implemented as at least one machine readable storage device. The at least one machine readable storage device comprising a plurality of instructions executable by circuitry of a controller for a fluid management system (FMS) configured to provide fluid flow to an endoscopic medical device cause the controller to implement any of the methods described herein.

In some embodiments, the disclosure can be implemented as a fluid management system (FMS) configured to provide fluid flow to an endoscopic medical device. The FMS can comprise a console configured to receive a fluid cassette, wherein the fluid cassette comprises a housing defining a fluid pathway therethrough, inflow tubing extending from the housing, the inflow tubing configured to couple to a source of fluid, and outflow tubing extending from the housing, the outflow tubing configured to couple to an endoscopic medical device, and the endoscopic medical device comprising an elongated shaft inserted into a lumen and configured to provide fluid from the outflow tubing to the lumen; a pump disposed in the console, the pump is configured to cause fluid to flow from the source of fluid through the fluid pathway; processing circuitry coupled to the pump; and memory coupled to the processing circuitry. The memory can store instructions that when executed by the processing circuitry cause the processing circuitry to receive an indication to operate the pump at a specified parameter, determine a predicted intraluminal pressure (ILP) of the lumen at a time step in the future, determine whether the predicted ILP is less than or equal to a threshold ILP level, and send, responsive to a determination that the predicted ILP is not less than or equal to the threshold ILP level, a first control signal to the pump, the first control signal to cause the pump to operate at a parameter different than the specified parameter, or send, responsive to a determination that the predicted ILP is less than or equal to the threshold ILP level, a second control signal to the pump, the second control signal to cause the pump to operate at the specified parameter.

With some embodiments of the FMS, the first control signal is configured to cause the pump to operate at a different RPM, to stop, or to reverse flow direction.

With some embodiments of the FMS, the memory can further store instructions, which when executed by the processing circuitry to receive the indication to operate the pump at the specified parameter, cause the processing circuitry to receive an indication to operate the pump at a specified RPM, wherein the first control signal is configured to cause the pump to operate at an RPM less than the specified RPM, and wherein the second control signal is configured to cause the pump to operate at the specified RPM.

With some embodiments of the FMS, the memory can further store instructions, which when executed by the processing circuitry, cause the processing circuitry to receive, from the pump, an indication of a current RPM of the pump; and determine the predicted ILP of the lumen at the time step in the future based in part on the current RPM of the pump and the specified RPM.

With some embodiments of the FMS, the memory can further store instructions, which when executed by the processing circuitry cause the processing circuitry to receive, from a medical device pressure sensor disposed in a distal end of the elongate shaft, an indication of a current ILP of the lumen; and determine the predicted ILP of the lumen at the time step in the future based in part on the current ILP of the lumen, the current RPM of the pump, and the specified RPM.

With some embodiments of the FMS, the console can comprise an FMS pressure sensor configured to measure a pressure in the fluid cassette, the memory can further store instructions, which when executed by the processing circuitry cause the processing circuitry to receive, from the FMS pressure sensor, an indication of a current pressure of the fluid cassette; and determine the predicted ILP of the lumen at the time step in the future based in part on the current pressure of the fluid cassette, the current ILP of the lumen, the current RPM of the pump, and the specified RPM.

With some embodiments of the FMS, the fluid pathway includes a fluid dampening chamber having a fluid inlet configured for fluid ingress into the fluid dampening chamber and a fluid outlet configured for fluid egress from the fluid dampening chamber, the memory further storing instructions, which when executed by the processing circuitry cause the processing circuitry to determine the predicted ILP of the lumen at the time step in the future based in part on fluid dynamics of the fluid dampening chamber.

In some embodiments, the disclosure can be implemented as a controller for a fluid management system (FMS) configured to provide fluid flow to an endoscopic medical device. The controller can comprise a memory storage device comprising instructions; and processing circuitry coupled to the memory and configured to receive an indication to operate a pump of the FMS at a specified parameter; determine a predicted intraluminal pressure (ILP) of a lumen at a time step in the future, wherein the endoscopic medical device comprises an elongated shaft inserted into the lumen and is configured to provide fluid from the FMS to the lumen; determine whether the predicted ILP is less than or equal to a threshold ILP level; and send, responsive to a determination that the predicted ILP is not less than or equal to the threshold ILP level, a first control signal to the pump, the first control signal to cause the pump to operate at a parameter different than the specified parameter; or send, responsive to a determination that the predicted ILP is less than or equal to the threshold ILP level, a second control signal to the pump, the second control signal to cause the pump to operate at the specified parameter.

With some embodiments of the controller, the memory storage device can further store instructions, which when executed by the processing circuitry cause the processing circuitry to solve a series of time-dependent differential equations; and determine the predicted ILP of the lumen at the time step in the future based on the solution to the series of time-dependent differential equations.

With some embodiments of the controller, the memory storage device can further store instructions, which when executed by the processing circuitry cause the processing circuitry to solve the series of time-dependent differential equations using a forward-Euler algorithm or a backward-Euler algorithm.

With some embodiments of the controller, the series of time-dependent differential equations relates a volume of the fluid cassette with the current ILP, relates fluid outflow resistance of the lumen to fluid inflow resistance of the lumen, and/or is based in part on compliance of the lumen.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates an example FMS in accordance with at least one embodiment of the present disclosure.

FIG. 1B illustrates the FMS of FIG. 1A in greater detail including a fluid cassette, in accordance with at least one embodiment of the present disclosure.

FIG. 1C, FIG. 1D, FIG. 1E, FIG. 1F, FIG. 1G, FIG. 1H, and FIG. 1I illustrate the fluid cassette of FIG. 1B is alternative views and detail.

FIG. 2A illustrates an endoscopic medical device that can be coupled to an FMS system, in accordance with at least one embodiment of the present disclosure.

FIG. 2B illustrates a workstation for the endoscopic medical device of FIG. 2A, in accordance with at least one embodiment of the present disclosure.

FIG. 3 illustrates an example of the endoscopic medical device of FIG. 2A and the FMS of FIG. 1A as may be used in a procedure, in accordance with at least one embodiment of the present disclosure.

FIG. 4 illustrates a routine to prevent ILP from exceeding a threshold level, in accordance with at least one embodiment of the present disclosure.

FIG. 5 illustrates a computing device that can be embodied in the FMS of FIG. 1A, in accordance with at least one embodiment of the present disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

Some fluid management systems (FMS) for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may regulate body cavity pressure when used in conjunction with an endoscope device such as, but not limited to, a LithoVue™ endoscope. The FMS can use pressure and/or temperature data from the endoscope or other endoscopic devices to adjust fluid flow and therefore regular pressure in the body cavity (e.g., intraluminal pressure (ILP)).

Direct regulation of ILP during a medical procedure may allow the fluid management system to safely drive system pressures of up to 600 mmHg to ensure no loss of flow during the procedure when tools are inserted into the working channel of the endoscope device. In some procedures, blood and/or debris may be present in the body cavity, which may negatively affect image quality through the endoscopic device. Fluid flow (e.g., irrigation) through the endoscopic device may be used to flush the body cavity to improve image quality. In some procedures, the body cavity may be relatively small and irrigation fluid may flow continuously, which can raise ILP and/or system pressure (e.g., fluid pressure within the fluid management system itself). As the volume of some cavities is very small and the irrigation fluid is continuously flowing into the cavity, the flow of fluid can cause high pressures in the cavity. Increased ILP and/or system pressure may pose risks to the patient under some circumstances.

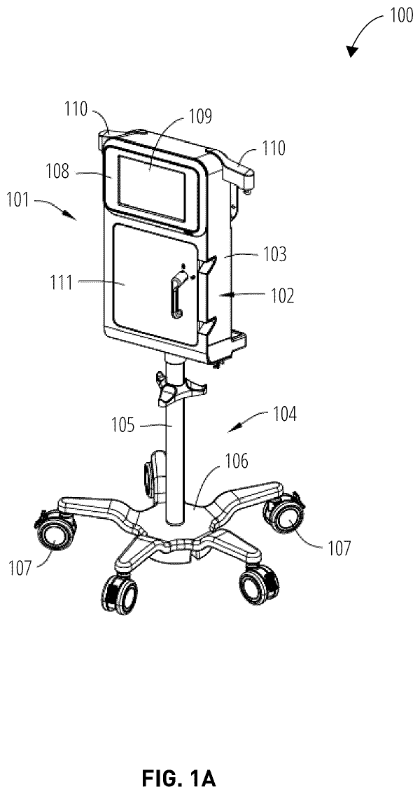

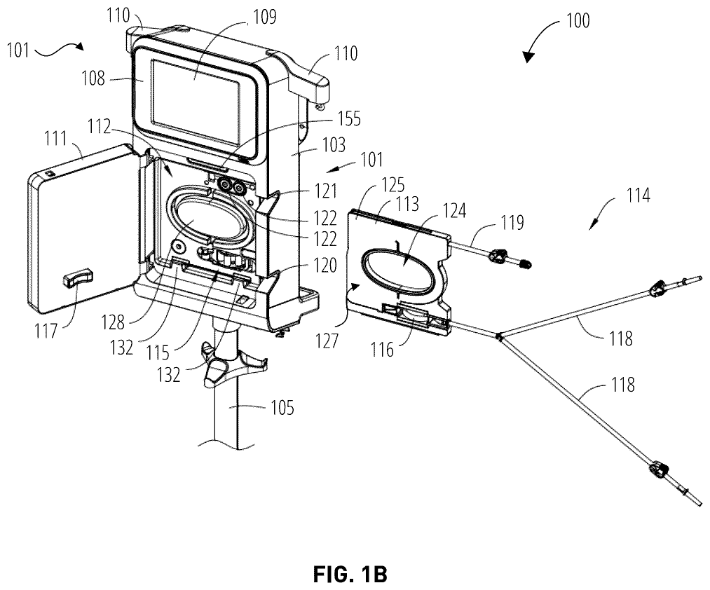

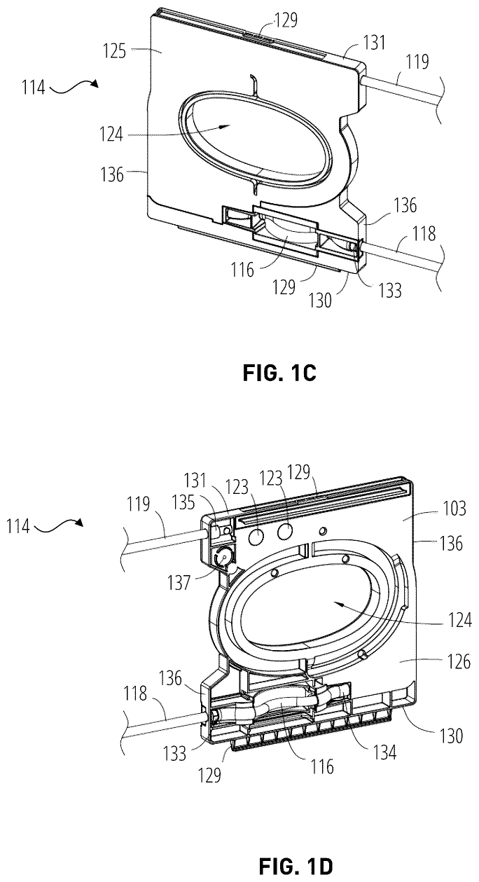

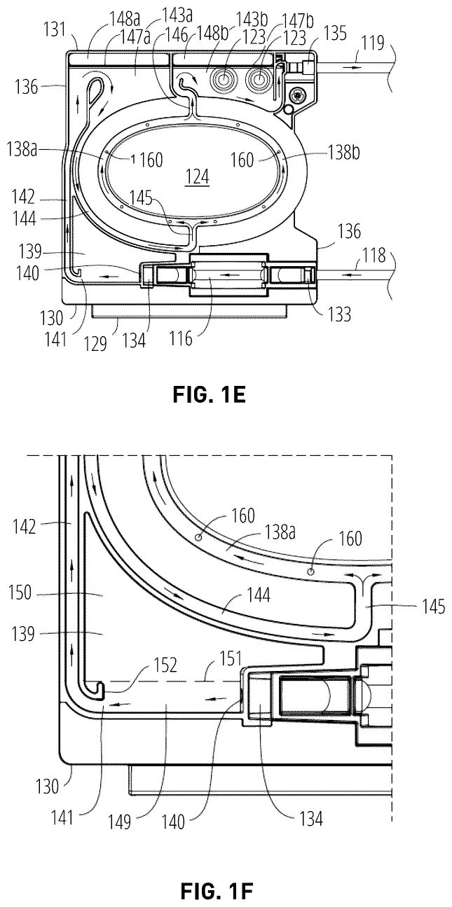

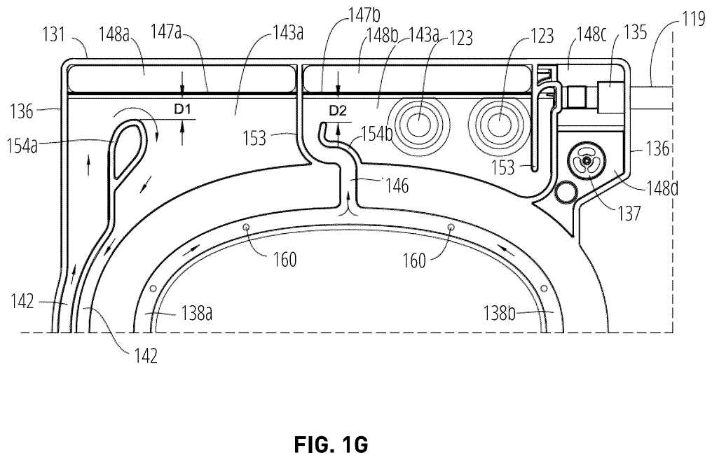

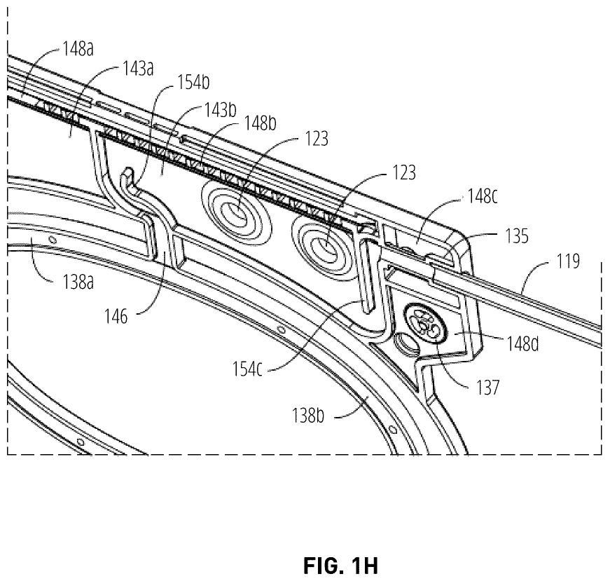

As noted, the present disclosure is directed to adjusting operation of the pump of an FMS prior to the ILP reaching a threshold limit to mitigate the risk or likelihood that the ILP will exceed the threshold limit. Accordingly, an example FMS is described to provide clarity in understanding the claimed embodiments. FIG. 1A illustrates an example fluid management system (FMS) 100 that may be used in an endoscopic procedure, such as fURS procedures while FIG. 1B illustrates a fluid cassette that can be inserted into and/or used with the FMS 100 to provide a flow of fluid to an endoscopic tool. FIG. 1C to FIG. 1I illustrates examples of the fluid cassette in various details.

Turning to FIG. 1A, a schematic view of an FMS 100 that may be used in an endoscopic procedure, such as fURS procedures is shown. The FMS 100 may be coupled to a medical device (not shown), such as an endoscope, that allows flow of fluid therethrough. In some instances, as detailed more fulling herein, the endoscope may include a pressure sensor to provide ILP feedback to the FMS 100. For example, the Litho Vue™ Elite endoscope includes a pressure sensor.

The FMS 100 includes a fluid management unit or console 101 including a controller 102 housed within a housing 103 of the console 101. In some instances, the console 101 may be portable and/or mobile such that the console 101 may be moved as desired. For instance, the console 101 may be mounted on a wheeled cart 104. For example, the wheeled cart 104 may include a pole 105 extending upward from a base 106. The base 106 may include a plurality of wheels 107 (e.g., caster wheels, or the like), allowing the cart 232 to be wheeled around to a desired location. In other instances, the console 101 may be provided with another form of cart, configured to be positioned on a flat surface, mounted to a wall, etc.

The FMS 100 may also include touch screen 108 including a display 109 and may further include switches or knobs in addition to touch capabilities. The touch screen 108 allows the user to input/adjust various functions of the FMS 100 such as, for example flow rate, pressure, and/or temperature. The user may also configure parameters and alarms (such as, but not limited to, a max pressure alarm, an ILP pressure alarm), information to be displayed, and the procedure mode. The touch screen 108 allows the user to add, change, and/or discontinue the use of various modular systems within the FMS 100. The touch screen 108 may also be used to change the FMS 100 between automatic and manual modes for various procedures. It is contemplated that other systems configured to receive user input may be used in place of or in addition to the touch screen 108, such as, but not limited to, voice commands.

The touch screen 108 may be configured to display, via display 109, a graphical user interface with selectable areas like buttons and/or may provide a functionality like physical buttons as would be understood by those skilled in the art. The display 109 may be configured to show icons related to modular systems and devices included in the FMS 100. The display 109 may also include graphical and/or textual indications of a fluid flow rate and/or fluid pressure. In some embodiments, operating parameters may be adjusted by touching a corresponding portion of the touch screen 108. The touch screen 108 may also display visual alerts and/or audio alarms if parameters (e.g., flow rate, temperature, etc.) are above or below predetermined thresholds and/or ranges. In some embodiments, the FMS 100 may also include further user interface components such as an optional foot pedal, a fluid warmer user interface, a fluid control interface, or other device to manually control various modular systems. For example, an optional foot pedal may be used to manually control flow rate.

The touch screen 108 may be operatively connected to or a part of the controller 102. The controller 102 includes at least processing circuitry and memory storing instructions that when executed by the processing circuitry, cause the controller 102 to behave as outlined herein. In some embodiments, the controller 102 can be a tablet computer, or other computing device. The controller 102 may be operatively connected to one or more system components such as, for example, an inflow pump, a fluid warming system, and a fluid deficit management system. In some embodiments, these features may be integrated into a single unit. The controller 102 is capable of and configured to perform various functions such as calculation, control, computation, display, etc.

The controller 102 is also capable of tracking and storing data pertaining to the operations of the FMS 100 and each component thereof. In some embodiments, the controller 102 may include wired and/or wireless network communication capabilities, such as ethernet or WIFI, through which the controller 102 may be connected to, for example, a local area network. The controller 102 may also receive signals from one or more of the sensors of the FMS 100. In some embodiments, the controller 102 may communicate with databases for best practice suggestions and the maintenance of patient records which may be displayed to the user on the display 109.

The fluid flow rate or the fluid pressure of fluid provided by the FMS 100 at any given time may be displayed on the display 109 to allow the operating room (OR) visibility for any changes. If the OR personnel notice a change in fluid flow rate or fluid pressure that is either too high or too low, the user may manually adjust the fluid flow rate or the fluid pressure back to a preferred level. The FMS 100 may also monitor and automatically adjust the fluid flow rate, or the fluid pressure based on previously set parameters, as discussed herein.

An illustrative console 101 may include one or more fluid container supports, such as fluid supply source hangers 110, each of which may support a fluid supply source (e.g., fluid bag). In some embodiments, placement and/or weight of the fluid supply source(s) hanging from the fluid supply source hangers 110 may be detected using a remote sensor and/or a supply load cell associated with and/or operatively coupled to each fluid supply source hanger 110 and/or fluid container support. The controller 102 may be in electronic communication with the supply load cell. The fluid supply source hangers 110 may be configured to receive a variety of sizes of the first fluid supply source(s) such as, for example, 1 liter (L) to 5 L fluid bags (e.g., saline bags). It will be understood that any number of fluid supply sources may be used. The fluid supply source hangers 110 may extend from the housing 103 of the console 101 and may include one or more hooks from which one or more fluid supply sources may be suspended. In some embodiments, the fluid used in the fluid management unit may be 0.9% saline. However, it will be understood that a variety of other fluids of varying viscosities, concentrations, mixtures, and/or consistencies may be used depending on the procedure.

Turning to FIG. 1B, the console 101 along with a fluid tubing set 114 configured to be coupled to a medical device (e.g., an endoscope, or the like) is depicted. As shown, the console 101 may include a door 111 hingedly attached to the housing 103. The door 111 may be opened to access a receptacle 112 configured to receive a fluid cassette 113 of the fluid tubing set 114 therein. In some examples, the fluid tubing set 114 is a single use medical device. The FMS 100 may include an inflow pump 115 configured to operatively engage the fluid tubing set 114 to pump and/or transfer fluid from a fluid supply source (e.g., a fluid bag, etc.) through the fluid tubing set 114 to a treatment site during a medical procedure. For example, the inflow pump 115 may be a roller pump or peristaltic pump positioned in the receptacle 112 configured to engage a length of flexible pump tubing 116 of the fluid cassette 113 when inserted therein. The door 111 may include an occlusion bed 117 mounted on the interior surface of the door 111. The occlusion bed 117 is configured to engage the length of flexible pump tubing 116 of the fluid cassette 113 when the door 111 is closed, to compress the length of flexible pump tubing 116 between the occlusion bed 117 and the inflow pump 115. The occlusion bed 117 may include a concave surface configured to engage the length of flexible pump tubing 116, which extends in an arcuate path around the inflow pump 115.

The inflow pump 115 may be electrically driven and may receive power from a line source such as a wall outlet, an external or internal electrical storage device such as a disposable or rechargeable battery, and/or an internal power supply. The inflow pump 115 may operate at any desired speed sufficient to deliver fluid at a desired pressure such as, for example, 5 mmHg to 50 mmHg, and/or at a target fluid flow rate or a target fluid pressure. As noted herein, the inflow pump 115 may be automatically adjusted based on, for example, pressure and/or temperature readings within the treatment site and/or visual feedback from the medical device attached thereto and inserted into the treatment site.

In some embodiments, the controller 102 may be configured to control the inflow pump 115 to maintain a target fluid flow rate or target fluid pressure based on a set of system operating parameters. Further, the controller 102 can be configured to control the inflow pump 115 to prevent the ILP from exceeding a selected threshold. With some embodiments, this can include identifying a prediction of the ILP reaching the selected threshold and stopping, reversing, or otherwise adjusting operation of the pump to prevent the ILP from exceeding the threshold value even after the pump stops.

The inflow pump 115 may also be manually adjusted via, for example, an optional foot pedal, the touch screen 108, voice commands, or a separate fluid controller. While not explicitly shown, the fluid controller may be a separate user interface including buttons that allow the user to increase or decrease the inflow pump 115. Alternatively, the fluid controller may be incorporated into the controller 102 and receive input via the touch screen 108, voice commands, or other means of input. It will be understood that any number of pumps may be used. In some embodiments, the FMS 100 may include multiple pumps having different flow capabilities. In some embodiments, a flow meter may be located before and/or after the inflow pump 115.

A housing 127 of the fluid cassette 113 may include an opening 124, such as an oval opening, extending through the housing 103 from the front face 125 to the rear front face 125. The opening 124 may extend most of the length of the housing 127 (i.e., most of the distance between the lateral edges of the housing 127) and/or most of the height of the housing 127 (i.e., most of the distance between the upper edge and the lower edge of the housing 127), in some instances. The opening 124 may be configured to receive an elevated portion of the rear wall of the receptacle 112, which may correspond to a fluid warming system 128. The elevated portion of the rear wall of the receptacle 112 may be an oval shape sized to fit through the oval shaped opening 124 of the housing 127 of the fluid cassette 113 when the fluid cassette 113 is in its loaded position in the receptacle 112.

In embodiments, in which the console 101 lacks a fluid warming system, the elevated portion of the rear wall of the receptacle 112 may still be present. Insertion of the elevated portion of the rear wall of the receptacle 112 through the opening 124 of the fluid cassette 113 may facilitate proper alignment of the fluid cassette 113 in the receptacle 112, for example.

The fluid tubing set 114 may include fluid inflow tubing 118 providing a fluid inflow from the fluid supply source into the interior of the fluid cassette 113. In some instances, the fluid inflow tubing 118 may include a bifurcated tubing with a first tubing section fluidly connected to a first fluid supply source and a second tubing section fluidly connected to a second fluid supply source. The first and second tubing sections may converge (such as at a Y-fitting) to a common tubing section extending to the fluid cassette 113. The end of the first tubing section and/or the second tubing section may include a bag spike, or other connector for connecting to the fluid supply source(s). The fluid tubing set 114 may also include fluid outflow tubing 119 providing a fluid outflow from the interior of the fluid cassette 113 to a medical device connected thereto. The fluid tubing set 114, including the fluid cassette 113, the fluid inflow tubing 118, and the fluid outflow tubing 119, may be disposable and provided sterile and ready to use.

When the fluid cassette 113 is installed in the receptacle 112 and the door 111 is closed, the fluid inflow tubing 118 may pass through a channel 120 extending through a wall of the housing 103 of the console 101 to an exterior of the console 101. Likewise, when the fluid cassette 113 is installed in the receptacle 112 and the door 111 is closed, the fluid outflow tubing 119 may pass through a channel 121 extending through a wall of the housing 103 of the console 101 to an exterior of the console 101. The channels 120 and 121 may both extend from the exterior of the console 101 to the receptacle 112. In some instances, both the channels 120 and 121 may be located on the same sidewall of the console 101 such that both the fluid inflow tubing 118 and the fluid outflow tubing 119 extend from the console 101 on the same side of the console 101.

The console 101 may further include one or more sensors, such as a pressure sensor and/or a bubble sensor. For instance, the console 101 may include a pressure sensor 122, illustrated as a pair of pressure sensors 122, configured to monitor a system pressure of fluid exiting the fluid cassette 113 and flowing through the fluid outflow tubing 119 to a surgical site. The fluid cassette 113 may include a corresponding pressure sensor interface 123, such as, for example, a flexible membrane (see FIG. 1C and FIG. 1D) that allow the pressure sensor 122 to monitor the pressure of fluid flowing through the fluid cassette 113 when the fluid cassette 113 is installed in the receptacle 112 of the console 101.

As outlined more fully below, the controller 102 can be configured to adjust the operation of the inflow pump 115 to regulate an ILP, and more specifically to control the parasitic timing, flow direction, and/or RPM s of the inflow pump 115 to prevent a ILP from exceeding a threshold level. This is described in greater detail below. However, in general, processing circuitry of the controller 102 can be configured to execute instructions to cause the controller to predict an ILP in the future. Further, the controller 102 can adjust the operation of the inflow pump 115, based on (or responsive to) the predicted ILP, to prevent or mitigate the actual ILP from exceeding the threshold level. For example, controller 102 can adjust the on/off state of the inflow pump 115, reverse flow direction of the inflow pump 115 (e.g., to partially depressurize the fluid tubing set 114, the fluid cassette 113, or the lumen), modulate the parasitic timing and/or RPMs of the pump 115, or the like.

To that end, example fluid pathways and fluid dynamics for the fluid cassette 113 are described with reference to FIG. 1C to FIG. 1I. It is noted that this description is not limiting to the overall claims but is provided for clarity in understanding the mechanisms between predicting a future ILP and moderating current operations of the inflow pump 115 in FMS 100 based on the predicted ILP to prevent the ILP from exceeding the threshold value.

Turning to FIG. 1C and FIG. 1D, the fluid cassette 113 is illustrated in greater detail. As depicted, the fluid cassette 113 may include one or more retention features configured to interact with the console 101 to retain the fluid cassette 113 in the receptacle 112 of the console 101. For example, the fluid cassette 113 may include one or more retention tabs 129 extending from a lower edge 130 of the housing 127 of the fluid cassette 113 and/or extending from an upper edge 131 of the housing 127 of the fluid cassette 113. The one or more retention tabs 129 are configured to engage or mate with one or more corresponding retention features 132 of the console 101. The console 101 may include a retention release mechanism, such as button 155, which may be actuated to release the fluid cassette 113 from the receptacle 112.

In some embodiments, the fluid cassette 113 may include a fluid inlet port 133 and a fluid outlet port 135 located at a lateral side of the fluid cassette 113 accessible from the first side edge 136 of the fluid cassette 113. The fluid inlet port 133 may be coupled to the fluid inflow tubing 118 and the fluid outlet port 135 may be coupled to the fluid outflow tubing 119, with the fluid inflow tubing 118 and the fluid outflow tubing 119 extending laterally from the first side edge 136.

The length of flexible pump tubing 116 of the fluid cassette 113, configured to engage and be compressed by the rollers of the inflow pump 115, may extend from the fluid inlet port 133 to a fluid connection 134 of the fluid cassette 113 leading to the fluid pathway defined through the interior of the fluid cassette 113. The flexible pump tubing 116 may be a discrete length of tubing separate from the fluid inflow tubing 118 and the fluid outflow tubing 119. In some instances, the flexible pump tubing 116 may extend through an arcuate pathway between the fluid inlet port 133 to the fluid connection 134, such that the flexible pump tubing 116 follows the rotational path of the rollers of the inflow pump 115. The fluid inlet port 133, the fluid outlet port 135, and/or the fluid connection 134 may be formed as a portion of the housing 127 of the fluid cassette 113 or formed separately and connected thereto.

The fluid cassette 113 may also include an air vent valve 137 configured to release air from the interior of the fluid cassette 113 to atmosphere. For example, the air vent valve 137 may include a hydrophobic membrane, allowing air, including bubbles entrained in the fluid, to pass through the hydrophobic membrane while preventing fluid within the fluid cassette 113 to pass therethrough. Air from within the fluid cassette 113 may then be vented to atmosphere through the air vent valve 137.

The internal flow pathway through the fluid cassette 113 is shown with arrows in the cross-sectional view of FIG. 1E and FIG. 1F. As depicted, fluid flows into the interior of the fluid cassette 113 through the fluid inlet port 133 from the fluid inflow tubing 118, and then passes through the flexible pump tubing 116 as the flexible pump tubing 116 is cyclically compressed by the rollers of the inflow pump 115. The fluid then flows into a fluid dampening fluid dampening chamber 139 configured to reduce pressure fluctuations of the pulsatile fluid flow exiting the flexible pump tubing 116 created by the inflow pump 115, and thus smoothen the fluid flow as the fluid exits the fluid dampening fluid dampening chamber 139. The fluid dampening fluid dampening chamber 139 may include a single fluid inlet 140 and a single fluid outlet 141. The fluid inlet 140 and the fluid outlet 141 may be located on opposite sides of the fluid dampening fluid dampening chamber 139, such that fluid flows into the fluid dampening fluid dampening chamber 139 through the fluid inlet 140 and flows out of the fluid dampening fluid dampening chamber 139 through the fluid outlet 141. More details of the fluid dampening chamber 130 will be described herein.

As fluid exits the fluid dampening fluid dampening chamber 139 through the fluid outlet 141 the fluid flows upward through the ascending fluid ascending fluid pathway 142. The ascending fluid pathway 142 interconnects the fluid dampening fluid dampening chamber 139 with a first air vent chamber 143a. The fluid then exits the first air vent chamber 143a in a downward direction along a descending fluid pathway 144 as shown in FIG. 1F. The descending fluid pathway 144 may be an arcuate pathway extending from an upper region above the opening 124 to a lower region below the opening 124.

The fluid may then enter a bifurcated fluid pathways 138a and 138b from the descending fluid pathway 144 as the fluid passes through a fluid warmer inlet channel 145 interconnecting the descending fluid pathway 144 and the bifurcated fluid pathways 138a and 138b. The bifurcated fluid pathways 138a and 138b includes a first fluid warming pathway (e.g., bifurcated fluid pathway 138a) extending from the fluid warmer inlet channel 145 in a first direction and a second fluid warming pathway (e.g., bifurcated fluid pathway 138b) extending from the fluid warmer inlet channel 145 in a second, generally opposite direction.

The bifurcated fluid pathway 138a may extend around a first portion of the opening 124 on a first side of the opening 124 and the bifurcated fluid pathway 138b may extend around a second portion of the opening 124 on a second, opposite side of the opening 124. The bifurcated fluid pathways 138a and 138b may then converge at a fluid mixing channel 146 located above the opening 124. Thus, the opening 124 may be located between the fluid mixing channel 146 and the fluid warmer inlet channel 145, such that the fluid mixing channel 146 is positioned above the opening 124 and the fluid warmer inlet channel 145 is positioned below the opening 124.

Fluid may flow upward from the fluid mixing channel 146 into a second air vent chamber 143b. The fluid may then exit the second air vent chamber 143b to the fluid outflow tubing 119 through the fluid outlet port 135.

It is noted that the bifurcated fluid pathways 138a and 138b are described herein as defining first and second fluid warming pathways. In instances in which the fluid cassette 113 and/or the console 101 include fluid warming capabilities, this is the region of the fluid pathway in which the fluid passing though the fluid cassette 113 may be warmed to an elevated temperature. However, in other instances in which the fluid cassette 113 and/or the console 101 does not include fluid warming capabilities, or in instances in which the fluid warming capabilities are disabled or deactivated (e.g., turned off), the bifurcated fluid pathways 138a and 138b may still be described as including a first fluid warming pathway and a second fluid warming pathway. Thus, describing the pathways as “warming” pathways will be used throughout this disclosure regardless of whether fluid is being warmed in the pathways. The bifurcated fluid pathway 138a may alternatively be referred to as a first branch of the bifurcated fluid pathway and the bifurcated fluid pathway 138b may alternatively be referred to as a second branch of the bifurcated fluid pathway. Thus, the bifurcated fluid pathway may split into a first branch and a second branch as the bifurcated fluid pathway passes around the opening 124.

A hydrophobic membrane 147a may be provided with each of the first and second air vent chambers 143a and 143b. For example, a first hydrophobic membrane 147a may be positioned at an interface between the first air vent chamber 143a and a first air chamber 148a. The interface including the first hydrophobic membrane 147a may be located along an upper extent of the first air vent chamber 143a such that air may pass through the first hydrophobic membrane 147a into the first air chamber 148a while the fluid in the first air vent chamber 143a is prevented from passing through the first hydrophobic membrane 147a. A second hydrophobic membrane 147b may be positioned at an interface between the second air vent chamber 143b and a second air chamber 148b. The interface including the second hydrophobic membrane 147b may be located along an upper extent of the second air vent chamber 143b such that air may pass through the second hydrophobic membrane 147b into the second air chamber 148b while the fluid in the second air vent chamber 143b is prevented from passing through the second hydrophobic membrane 147b.

During usage of the fluid cassette 113, a volume of fluid 149 may fill the lower portion of the fluid dampening chamber 139 while a volume of air 150 is trapped in the upper portion of the fluid dampening chamber 139. The fluid level 151 is the direct interface between the volume of fluid 149 and the volume of air 150. The fluid dampening chamber 139 may be designed to substantially smoothen the pulsatile fluid flow from the inflow pump 115 for fluid flows up to 800 ml/min, in some instances. For example, it has been found that sizing the fluid dampening chamber 139 such that the volume of air 150 is at least 38 ml substantially smoothens the pulsatile fluid prior to exiting the fluid dampening chamber 139. Accordingly, the fluid dampening chamber 139 may be sized to provide a volume of air 150 of 38 ml or more, or 40 ml or more, in some instances. For instance, the fluid dampening chamber 139 may be sized to provide a volume of air 150 of 38 ml to 42 ml, during use.

Furthermore, as noted above, the fluid dampening chamber 139 may include a single fluid inlet 140 and a single fluid outlet 141 located on opposite sides of the fluid dampening chamber 139, such that fluid flows into the fluid dampening chamber 139 through the fluid inlet 140 and flows out of the fluid dampening chamber 139 through the fluid outlet 141. The fluid inlet 140 and the fluid outlet 141 may be positioned near a base of the fluid dampening chamber 139. The fluid dampening chamber 139 may be configured such that the upper extent of the fluid outlet 141 is lower (i.e., closer to the lower edge 130 of the fluid cassette 113) than the upper extent of the fluid inlet 140. This ensures that the fluid level 151 is above the upper extent of the fluid outlet 141 such that air from the volume of air 150 is not pulled out of the fluid dampening chamber 139 into fluid exiting the fluid dampening chamber 139 though the fluid outlet 141 into the ascending fluid pathway 142, which could otherwise occur at high flow rates. In some instances, the fluid outlet 141 may include a lip 152 extending upward from the upper extent of the opening of the fluid outlet 141 into the fluid dampening chamber 139. The lip 152 may have any desired height. In some instances, the height of the lip 152 may be sized such that the fluid level 151 is above the upper extent of the lip 152. In other instances, the fluid level 151 may impinge the lip 152.

As shown in FIG. 1G, the first air chamber 148a may be interconnected with the second air chamber 148b, and the combination for the air chambers 148a and 148b may vent air to atmosphere through the air vent valve 137. For example, the air in the first air chamber 148a and the second air chamber 148b may pass into a third air chamber 148c defined as a cavity of the housing 127 of the fluid cassette 113 surrounding the fluid outlet port 135. In turn, the third air chamber 148c may be connected to a fourth air chamber 148d defined as a cavity of the housing 127 of the fluid cassette 113 in direct communication with the air vent valve 137. Accordingly, air may be vented from the first air chamber 148a and/or the second air chamber 148b through the air chambers 148c and 148d to exit the fluid cassette 113 via the air vent valve 137.

A wall 153 may be located between and separating the first air vent chamber 143a from the second air vent chamber 143b such that the first air vent chamber 143a is not directly fluidly connected to the second air vent chamber 143b, but rather the first air vent chamber 143a is in fluid communication with the second air vent chamber 143b only via the bifurcated fluid pathways 138a 138b.

The first air vent chamber 143a may include a deflector 154a, configured as an interior wall within the first air vent chamber 143a, that helps direct fluid flowing upward from the ascending fluid flow ascending fluid pathway 142 toward the upper extent of the first air vent chamber 143a and the first hydrophobic membrane 147a. An upper extent of the deflector 154a may be located closer to the upper extent of the first air vent chamber 143a than to a lower extent of the first air vent chamber 143a. In some instances, the upper extent of the deflector 154a may be located within 0.8 inches or less, within 0.7 inches or less, within 0.6 inches or less, within 0.5 inches or less, or within 0.4 inches or less of the upper extent of the first air vent chamber 143a and the first hydrophobic membrane 147a. Accordingly, the distance D₁ between the upper extent of the deflector 154a and the first hydrophobic membrane 147a may be in the range of about 0.3 inches to about 0.7 inches, in the range of about 0.3 inches to about 0.5 inches, or in the range of about 0.35 inches to about 0.4 inches, for example.

The second air vent chamber 143b may include a deflector 154b, configured as an interior wall within the second air vent chamber 143b, that helps direct fluid flowing upward from the fluid mixing channel 146 toward the upper extent of the second air vent chamber 143b and the second hydrophobic membrane 147b. An upper extent of the deflector 154b may be located closer to the upper extent of the second air vent chamber 143b than to a lower extent of the second air vent chamber 143b. In some instances, the upper extent of the deflector 154b may be located within 0.8 inches or less, within 0.7 inches or less, within 0.6 inches or less, within 0.5 inches or less, or within 0.4 inches or less of the upper extent of the second air vent chamber 143b and the second hydrophobic membrane 147b. Accordingly, the distance D₂ between the upper extent of the deflector 154b and the second hydrophobic membrane 147b may be in the range of 0.3 inches to about 0.7 inches, in the range of about 0.3 inches to about 0.5 inches, in the range of about 0.4 inches to about 0.5 inches, or in the range of about 0.4 inches to about 0.45 inches, for example.

As shown in FIG. 1H, the second air vent chamber 143b may be configured with the fluid inlet into the second air vent chamber 143b from the fluid mixing channel 146 above (i.e., closer to the upper edge 131 of the fluid cassette 113) the fluid outlet from the second air vent chamber 143b into the fluid outlet port 135. For example, the second air vent chamber 143b may also include a deflector 154c defining a fluid outlet from the second air vent chamber 143b to the outflow fluid outlet port 135. The deflector 154c may position the fluid inlet into the second air vent chamber 143b higher (i.e., closer to the upper edge 131 of the fluid cassette 113) than the fluid outlet from the second air vent chamber 143b. Such as configuration may facilitate fluid flow adjacent to the second hydrophobic membrane 147b prior to fluid exiting the second air vent chamber 143b.

The pressure sensor interfaces 123 may be in a wall of the second air vent chamber 143b such that the pressure sensors 122, discussed above, can monitor the fluid pressure of the fluid within the fluid cassette 113 just prior to the fluid exiting the fluid cassette 113. For instance, the pressure sensor interfaces 123 may be a flexible membrane that flexes against the pressure sensors on an exterior thereof, as the pressure of the fluid within the second air vent chamber 143b impinges upon the interior surface of the flexible membrane. The fluid pressure monitored may be considered a system pressure, for example, which may be utilized by the controller 102 to adjust the inflow pump 115 to maintain a desired pressure during a medical procedure.

FIG. 1I illustrates an exploded view of the fluid cassette 113. As depicted, the housing 127 of the fluid cassette 113 may be formed of multiple components, which when assembled form the fluid cassette 113. For example, the housing 127 may include a base 156 and a cover 157. The base 156 may include a plurality of interior walls defining the fluid pathway through the interior of the fluid cassette 113. The cover 157 may extend across the interior walls. In instances in which the fluid cassette 113 includes fluid warming capabilities, the fluid cassette 113 may include a stack of heating plates 158.

The stack of heating plates 158 may include a plurality of annular plates 159 stacked one on top of the other. The annular plates 159, which may be ring-shaped, may be formed of a metal material, such as stainless steel, for example. In some instances, the annular plates 159 may be circular or oval plates, with a central opening. Each of the plates 159 may include a flat upper surface and a flat lower surface, opposite the upper surface. The annular plates 159 may be stacked on top of each other, such that each plate 159 is spaced apart from adjacent plates to allow fluid to flow there between. In other words, the annular plates 159 may be configured such that there is a gap between the facing surfaces of the plurality of plates 159 (i.e., the upper surface of one plate 159 and the lower surface of a second, adjacent plate 159) to allow fluid to flow between the adjacent plates 159. For instance, the annular plates 159 may include, spacers, such as dimples 161 extending from one of the flat surfaces of the annular plates 159 (i.e., the upper and/or lower flat surfaces). The dimples 161, or other type of spacers, may be intermittently arranged around the perimeter of the annular plates 159 and configured to contact an upper/lower surface of an adjacent plate 159, retaining a gap between the adjacent plates 159 for fluid flow.

The stack of heating plates 158 may be disposed within the bifurcated fluid pathways 138a and 138b such that fluid passes directly across the stack of heating plates 158 as the fluid passes through the bifurcated fluid pathways 138a and 138b to transfer heat from the stack of heating plates 158 to the fluid. Furthermore, as shown in FIG. 1I, the interior surface of the base 156 and/or the cover 157 may include one or more spacers extending therefrom to contact a plate 159 and space the plate 159 apart from the interior surface of the base 156 and/or the cover 157. For instance, the spacers may be projections 160 extending into the bifurcated fluid pathways 138a and 138b to space the uppermost plate 159 of the stack of heating plates 158 from the interior surface of the cover 157 and/or the lowermost plate 159 of the stack of heating plates 158 from the interior surface of the base 156 of the housing 103 (see FIG. 1E to FIG. 1G).

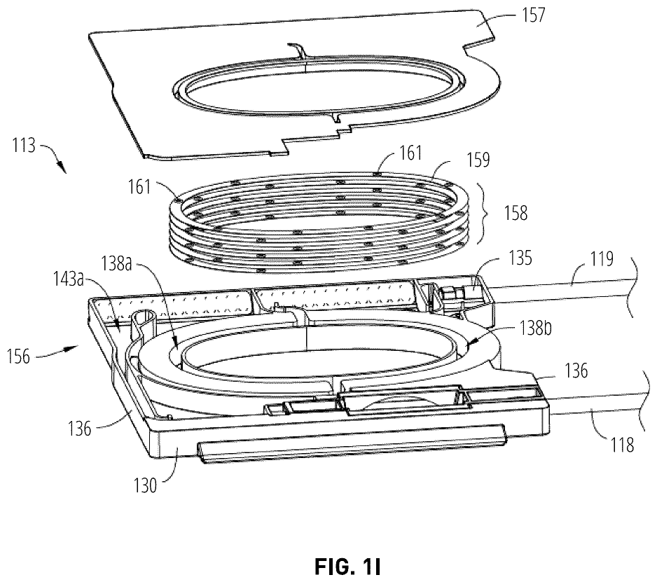

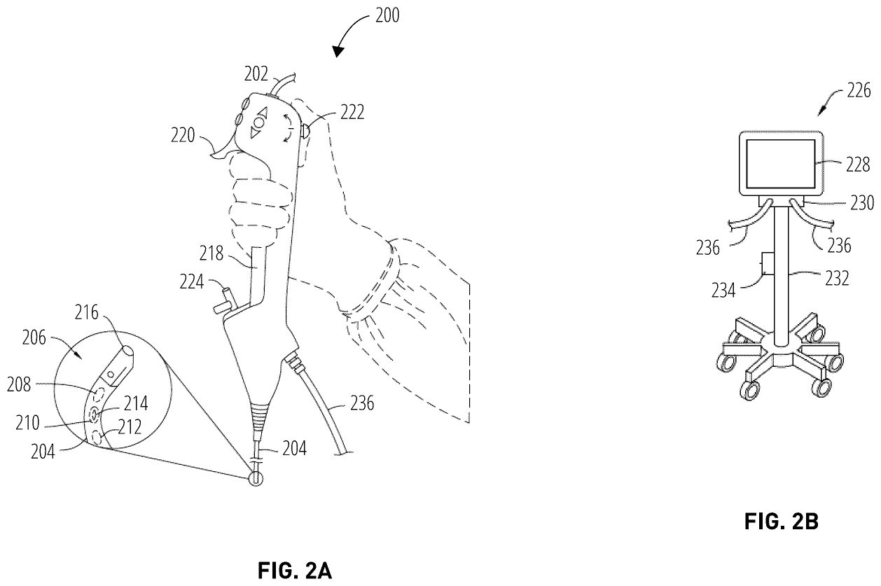

As introduced above, the FMS 100 can be fluidly and communicatively coupled to a medical device, such as, for example, the LithoVue™ Elite endoscope during a medical procedure (e.g., ureteroscopy, or the like). An example medical device 200 is depicted in FIG. 2A and FIG. 2B. The medical device 200 can be fluidly coupled to the FMS 100 via supply line 202. For example, supply line 202 can be configured to fluidly connect with fluid outflow tubing 119. The medical device 200 can receive a flow of fluid from FMS 100 via the fluid outflow tubing 119 and deliver the flow of fluid to a procedure site via an elongate elongated shaft 204. The elongated shaft 204 may include one or more working lumens for receiving a flow of fluid from the FMS 100 as well as working lumens for receiving other devices therethrough (e.g., an optical fiber, or the like). In some embodiments, the one or more supply lines 202 fluidly coupling the FMS 100 to the medical device 200 may be formed of a material the helps dampen the peristaltic motion created by the inflow pump 115. In some embodiments, the supply lines 202 may be formed from small diameter tubing less than or equal to 1/16 inches (1.5875 millimeters) in diameter. However, it will be understood that tubing size may vary based on the application. The supply lines 202 and/or the tubing may be disposable and provided sterile and ready to use. Different types of tubing may be used for various functions within the FMS 100. For example, one type of tubing may be used for fluid heating and fluid flow control to the medical device 200 while another type of tubing may be used for irrigation within the body and/or the treatment site.

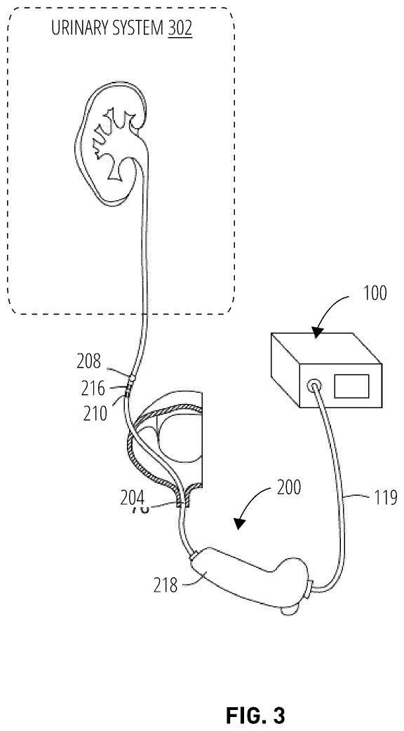

The medical device 200 may include one or more sensors proximate a distal end 206 of the elongate elongated shaft 204. For example, the medical device 200 may include a pressure sensor 208 at the distal end 206 of the elongate elongated shaft 204 to measure ILP within the treatment site (see FIG. 3). The medical device 200 may also include other sensors such as, for example, a temperature sensor 210, a Fiber Bragg grating optical fiber 212 to detect stresses, and/or an antenna or electromagnetic sensor 214 (e.g., a position sensor).

In an illustrative embodiment, the distal end 206 of the medical device 200 may also include at least one camera 216 to provide a visual feed to the user on a display screen (e.g., display 109 or another display coupled to the medical device 200).

The medical device 200 includes a handle 218 coupled to a proximal end of the elongate elongated shaft 204. The handle 218 may have a fluid flow on/off switch 220, which allows the user to control when fluid is flowing through the medical device 200 and into the treatment site. The handle 218 may further include other buttons 222 that perform other various functions. For example, in some embodiments, the handle 218 may include buttons 222 to control the temperature of the fluid. In some embodiments, the medical device 200 may also include a drainage port 224, which may be connected to a drainage system.

The medical device 200 may be in electronic communication with the FMS 100 and/or a dedicated workstation for the medical device 200. For example, medical device 200 can be connected to workstation 226 via a wired connection 236. It is noted that medical device 200 could, in some embodiments, be wirelessly connected to the workstation 226. The workstation 226 may include a computer 228 (e.g., tablet, touch screen computing device, or the like), an interface 230 for receiving the wired connection 236, a cart 232, and a power supply 234, among other features. In some embodiments, the interface 230 may be configured with a wired or wireless communication connection 236 for communicative coupling with the console 101 of the FMS 100. The computer 228 may include at least a display screen, a processor, and memory storing instructions executable by the processor. In some embodiments, the workstation 226 may be a multi-use component (e.g., used for more than one procedure) while the medical device 200 may be a single use device, although this is not required. In some embodiments, the workstation 226 may be omitted and the medical device 200 may be electronically coupled directly to the console 101 of the FMS 100.

As introduced above, an FMS (e.g., FMS 100, or the like) for use in fURS procedures (e.g., ureteroscopy, percutaneous nephrolithotomy (PCNL), benign prostatic hyperplasia (BPH), transurethral resection of the prostate (TURP), etc.), gynecology, and other endoscopic procedures may regulate body cavity pressure when used in conjunction with an endoscope device such as, the medical device 200. Direct regulation of the ILP during a medical procedure may allow the FMS 100 to safely drive system pressures of up to 600 mmHg to ensure no loss of flow during the procedure when tools are inserted into the working channel of the medical device 200. In some procedures, blood and/or debris may be present in the body cavity, which may negatively affect image quality through the endoscopic device. Fluid flow (e.g., irrigation) through the medical device 200 may be used to flush the body cavity to improve image quality. In some procedures, the body cavity may be relatively small (e.g., a kidney) and irrigation fluid may flow continuously, which can raise ILP above threshold levels.

For example, FIG. 3 illustrates the FMS 100 fluidly coupled to the medical device 200. In this example, the medical device 200 may be a ureteroscope and may be used in a ureteroscopy procedure to access a patient's kidney. In such a procedure, the medical device 200, and particularly, the elongate elongated shaft 204 of the medical device 200 is inserted into the bladder and ureter and is used to diagnose and/or treat a variety of problems in the urinary tract. It will be understood that while FIG. 3 illustrates an exemplary embodiment of a ureteroscope in use, the features detailed herein may also be directly integrated into a cystoscope, an endoscope, a hysteroscope, or virtually any device with an image and fluid flow capability.

During use, the FMS 100 is repeatedly called on to provide fluid flow to the distal end 206 of the elongated shaft 204, for example to clear the visual field of the camera 216. In some embodiments, the inflow pump 115 is configured to operate in a “boost” mode, for example, to quickly provide the periodically requested fluid flow. In boost mode, the inflow pump 115 is operated at a highly elevated RPM, which is necessary to overcome the pressure in the fluid cassette 113 and the fluid outflow tubing 119, which can often include tens of feet of small diameter tubing. However, this elevated RPM can lead to the ILP increasing beyond the threshold level.

Further, as outlined above, even where the inflow pump 115 stops, the internal fluid dynamics of the FMS 100 (e.g., pressure in fluid cassette 113 and/or fluid outflow tubing 119, etc.) the ILP may continue to rise. Further, as outlined above, the ILP may continue to rise even after the inflow pump 115 is stopped. Given the delicate anatomy in which the elongated shaft 204 is inserted, ILP that exceed a threshold level may cause damage the patient. For example, exceeding safe ILP levels can result in damage to the renal system, excess fluid absorption, infection, pyelovenous backflow, among other complications.

As such, the present disclosure provides to predict the ILP at a future time and dynamically modulate operating parameters of the inflow pump 115 prior to the ILP reaching the threshold limit to mitigate the risk that the ILP will reach or exceed the threshold limit. Said differently, the present disclosure provides to stop, reduce speed, adjust timing, reverse flow direction, or otherwise adjust the operating parameters of the inflow pump 115 based on a prediction of the ILP at a future time to mitigate the risk that the ILP will exceed a threshold level.

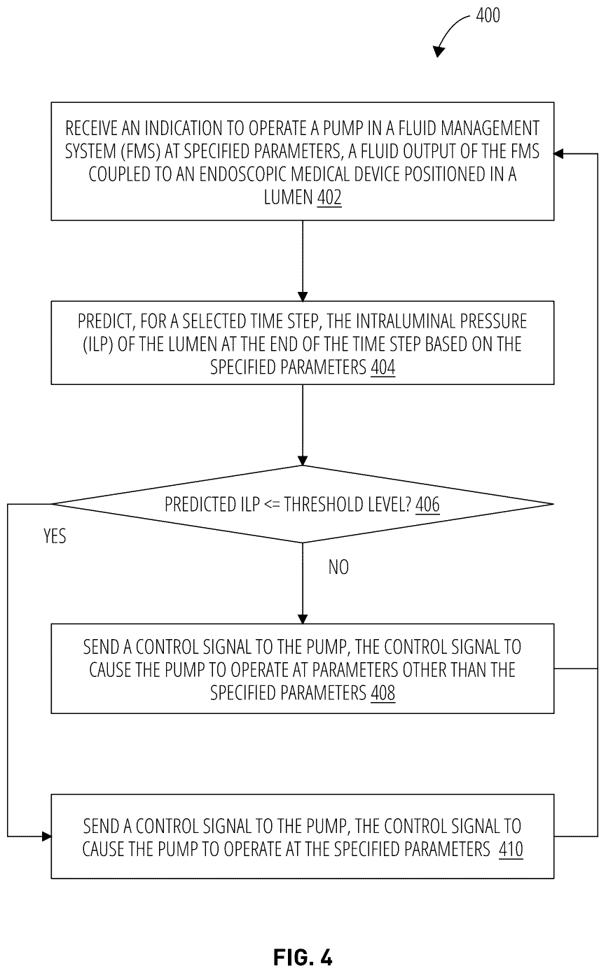

FIG. 4 illustrates a routine 400 for preventing ILP from exceeding a threshold level even after stopping a pump in an FMS system. With some embodiments, routine 400 can be implemented by FMS 100. Routine 400 can begin at block 402. At block 402 “receive an indication to operate a pump in an FMS at specified parameters, a fluid output of the FMS coupled to an endoscopic medical device positioned in a lumen” an indication to operate the inflow pump 115 of the FMS 100 at specified parameters (e.g., RPM, timing, etc.) is received at block 402. For example, processing circuitry of controller 102 can receive (e.g., from switch 220, or the like) an indication to operate the inflow pump 115 at a specified RPM and/or at specified parasitic timing. For example, when the switch 220 is initiated, the inflow pump 115 may be operated at a “boost” RPM to overcome the pressure in the fluid cassette 113 and fluid outflow tubing 119. As another example, when the switch 220 is continually depressed, the inflow pump 115 may operate at a lower RPM as the current flow rate may be sufficient.

It is noted that the specified parameters (e.g., RPM and/or timing) of the inflow pump 115 needed to provide the requested fluid flow from the FMS 100 depends upon a variety of factors. For example, current flow rate of the system as well as pressure within the fluid cassette 113 (e.g., as measured by pressure sensor 122, or the like) are related. With some embodiments, the pressure and flow rate are linearly related (e.g., for low flow rates) while in other embodiments, the pressure and flow rate are non-linearly related (e.g., for high flow rates). Further, the fluid volume (e.g., volume of fluid 149) within the fluid cassette 113 affects the operating parameters of the pump needed to provide the requested fluid flow.

Continuing to block 404 “predict, for a selected time step, the ILP of the lumen at the end of the time step based on the specified parameters” the ILP of the lumen in which the elongated shaft 204 of the medical device 200 is inserted is predicted at a future time, based in part on the operating parameters specified at block 402. For example, processing circuitry of controller 102, in executing instructions stored on a computer-readable medium, can determine an ILP of the lumen at a future time, or after a selected time step. In some examples, the time step can be 2 seconds(s), 2.5 s, 3 s, or 3.5 s. In some examples, the time step can be between greater than or equal to 3 s and less than or equal to 3.5 s, greater than or equal to 1 s and less than or equal to 10 s. With some examples, controller 102 determines the ILP at the end of the time step based on current conditions of the FMS 100 and the medical device 200 as well as the parameters specified at block 402. For example, controller 102 can receive indications of the current inflow pump 115 RPM, the current pressure within the fluid cassette 113 (e.g., based on measurements from pressure sensor 122, or the like), the current ILP (e.g., based on measurements from the pressure sensor 208, or the like) as well as requested future conditions of the system (e.g., requested inflow pump 115 RPM, or the like).

Given the above system conditions, processing circuitry of the controller 102 can execute instructions to determine the ILP at the end of the time step. For example, the controller 102 can solve a series of time-dependent differential equations. In some embodiments, controller 102 can execute instructions to solve the series of time-dependent differential equations using, for example, forward-Euler methods, backward Euler methods, Runge Kutta methods, A dams-Moulton methods, A dams-Bashforth methods, Hermite-Obreschkoff methods, Fehlberg methods, Parker-Sochacki methods, Bychkov-Scherbakov methods, Nyström methods, and/or Parallel-in-Time methods.

With some examples, one of the time-dependent equations relates the volume of fluid in the fluid cassette 113 with the current ILP. With some examples, one of the time-dependent equations relates fluid outflow resistance (e.g., resistance to fluid outflow from the lumen) to fluid inflow resistance (e.g., resistance to fluid inflow to the lumen). With some embodiments, the fluid inflow resistance is based on assumptions of the compliance of the lumen, which can be elastic.

Continuing to decision block 406 “predicted ILP<=threshold level?” a determination is made as to whether the predicted ILP at the end of the time step is less than or equal to a threshold level. It is noted that as outlined above, the threshold level can be set by the user of the FMS 100 or in some examples, the threshold level can be set based on a preselected procedure. At decision block 406, processing circuitry of controller 102 can execute instructions to determine whether the predicted ILP exceeds the threshold level. Routine 400 can continue from decision block 406 to either block 408 or block 410. Routine 400 can continue from decision block 406 to block 408 based on a determination that the predicted ILP is not less than or equal to the threshold level while routine 400 can continue from decision block 406 to block 410 based on a determination that the predicted ILP is less than or equal to the threshold level.

At block 408 “send a control signal to the pump, the control signal to cause the pump to operate at parameters other than the specified parameters” the controller 102 can send a control signal to the inflow pump 115 to cause the inflow pump 115 to operate at parameters different than specified at block 402. For example, if the inflow pump 115 is operating in or requested to be operating in boost mode but the ILP is predicted to exceed the threshold level, then controller 102 can send a control signal to the inflow pump 115 to cause the inflow pump 115 to operate at an RPM less than a boost mode RPM. In some examples, the controller 102 can send a control signal to the inflow pump 115 to cause the inflow pump 115 to stop where the ILP is predicted to exceed the threshold level. As another example, the controller 102 can send a control signal to the inflow pump 115 to cause the inflow pump 115 to reverse directions to partially depressurize the lumen, fluid cassette 113, and/or fluid tubing set 114 where the ILP is predicted to exceed the threshold level. In some examples, the action with which the controller 102 instructs, via the control signal, the inflow pump 115 to take (e.g., reduce RPM, stop, reverse, or the like) can be based on a differential between the predicted ILP and the threshold level. For example, the more the predicted ILP exceeds the threshold level the greater than actions with which controller 102 can signal to inflow pump 115. As a specific example, where the predicted ILP exceeds the threshold by a secondary threshold amount the controller 102 can send a control signal to the inflow pump 115 to cause the inflow pump 115 to reverse flow direction to depressurize (at least partially) the fluid tubing set 114, the fluid cassette 113, and/or the lumen. Alternatively, where the predicted ILP exceeds the threshold by less than the secondary threshold amount, the controller 102 can send a control signal to the inflow pump 115 to cause the inflow pump 115 to merely reduce RPM s.

At block 410 “send a control signal to the pump, the control signal to cause the pump to operate at the specified parameters” the controller 102 can send a control signal to the inflow pump 115 to cause the inflow pump 115 to operate at the parameters specified at block 402 (e.g., at the requested RPM, or the like).

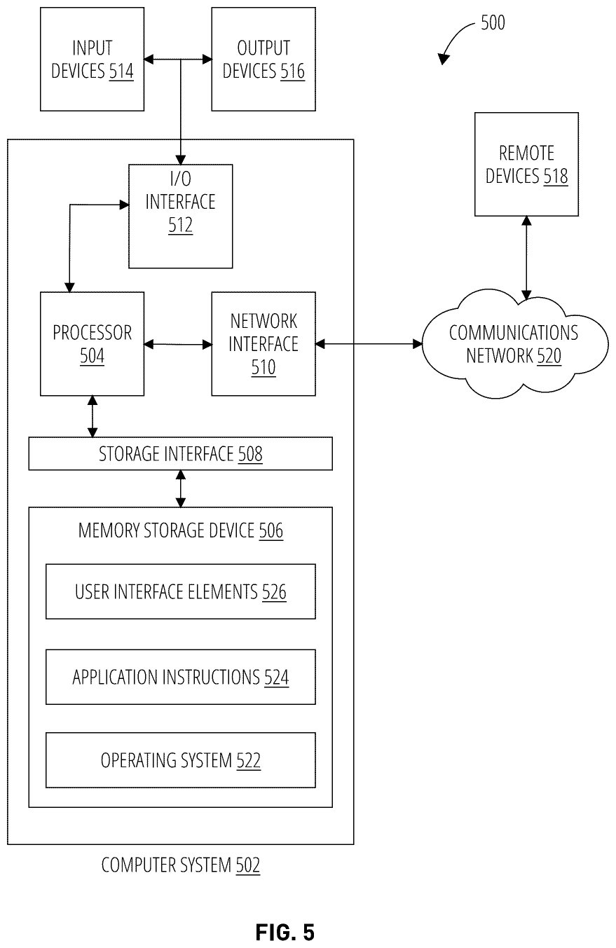

FIG. 5 is a block diagram of a computing environment 500 including a computer system 502 for implementing embodiments consistent with the present disclosure. In some embodiments, the computing environment 500, or portion thereof (e.g., the computer system 502) may comprise or be comprised in an FMS, such as FMS 100. For example, controller 102 can comprise the computing environment 500, or portions thereof (e.g., computer system 502, or the like). Accordingly, in various embodiments, computer system 502 may determine an ILP of a lumen at a future time based on current conditions of the FMS 100 and control the inflow pump 115 (e.g., allow RPM s as requested, reduce RPM s, stop the pump) of the FMS 100 to mitigate the risk that the ILP will exceed a threshold level in the future, even after the inflow pump 115 is stopped.

The computer system 502 may include processing circuitry, such as, for example, a central processing unit (“CPU” or “processor”) 504. The processor 504 may include at least one data processor for executing instructions and/or program components for executing user or system-generated processes. A user may include a person, a person using a device such as those included in this disclosure, or another device. The processor 504 may include specialized processing units such as integrated system (bus) controllers, memory management control units, floating point units, graphics processing units, neural processing units, digital signal processing units, etc. The processor 504 may be disposed in communication with input devices 514 and output devices 516 via I/O interface 512. The I/O interface 512 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular, etc.

Using the I/O interface 512, computer system 502 may communicate with input devices 514 and output devices 516. In some embodiments, the processor 504 may be disposed in communication with a communications network 520 via a network interface 510. In various embodiments, the communications network 520 may be utilized to communicate with a remote memory storage device 506, such as for accessing look-up tables, performing updates, or utilizing external resources. The network interface 510 may communicate with the communications network 520. The network interface 510 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 520 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 520 may include a variety of network devices, including routers, bridges, servers, computing devices, storage devices, etcetera. In some embodiments, the processor 504 may be disposed in communication with a memory storage device 506 via a storage interface 508. The storage interface 508 may connect to memory storage device 506 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 506 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 506 may store a collection of program or database components, including, without limitation, an operating system 522, an application instructions 524, and a user interface elements 526.

In various embodiments, the operating system 522 may facilitate resource management and operation of the computer system 502. Examples of operating systems include, without limitation, APPLE MACINTOSH® OS X®, UNIX®, UNIX-like system distributions (E.G., BERKELEY SOFTWARE DISTRIBUTION® (BSD), FREEBSD®, NETBSD®, OPENBSD, etc.), LINUX DISTRIBUTIONS (E.G., RED HAT®, UBUNTU®, KUBUNTU®, etc.), IBM®OS/2ª, MICROSOFT® WINDOWS® (XP, VISTA®/7/8, 10 etc.), APPLE® IOS®, GOOGLE™ ANDROID™, BLACKBERRY® OS, or the like.

The application instructions 524 may include instructions that when executed by the processor 504 cause the processor 504 to perform one or more techniques, steps, procedures, and/or methods described herein, such to, for example, operations associated with routine 400. To that end, memory storage device 506 may store data associated with ILP measurements, ILP threshold levels, fluid cassette 113 pressure measurements, fluid cassette 113 fluid volume, inflow pump 115 RPM, FMS 100 system flow rate, etc.

The user interface elements 526 may facilitate display, execution, interaction, manipulation, or operation of program components through textual or graphical facilities. For example, user interfaces may provide computer interaction interface elements on a display system operatively connected to the computer system 502, such as cursors, icons, checkboxes, menus, scrollers, windows, widgets, etcetera. The user interface elements 526 may be employed by application instructions 524 and/or operating system 522 to provide, for example, a user interface with which a user can interact with computer system 502. In some embodiments, the user interface elements 526 may be integrated with the display (e.g., display 109).

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A fluid management system (FMS) to provide fluid flow to an endoscopic medical device, the FMS comprising:
a console configured to receive a fluid cassette,
wherein the fluid cassette comprises a housing defining a fluid pathway therethrough, inflow tubing extending from the housing, the inflow tubing configured to couple to a source of fluid, and outflow tubing extending from the housing, the outflow tubing configured to couple to an endoscopic medical device, and
wherein the endoscopic medical device comprises an elongated shaft inserted into a lumen and configured to provide fluid from the outflow tubing to the lumen;
a pump disposed in the console, wherein the pump is configured to cause fluid to flow from the source of fluid through the fluid pathway;
processing circuitry coupled to the pump; and
memory coupled to the processing circuitry, the memory storing instructions that when executed by the processing circuitry cause the processing circuitry to:
receive an indication to operate the pump at a specified parameter,
determine a predicted intraluminal pressure (ILP) of the lumen at a time step in the future,
determine whether the predicted ILP is less than or equal to a threshold ILP level, and
send, responsive to a determination that the predicted ILP is not less than or equal to the threshold ILP level, a first control signal to the pump, the first control signal to cause the pump to operate at a parameter different than the specified parameter, or
send, responsive to a determination that the predicted ILP is less than or equal to the threshold ILP level, a second control signal to the pump, the second control signal to cause the pump to operate at the specified parameter.

2. The FMS of claim 1, wherein the first control signal is configured to cause the pump to operate at a different RPM, to stop, or to reverse flow direction.

3. The FMS of claim 1, the memory further storing instructions, which when executed by the processing circuitry to receive the indication to operate the pump at the specified parameter, cause the processing circuitry to:
receive an indication to operate the pump at a specified RPM,
wherein the first control signal is configured to cause the pump to operate at an RPM less than the specified RPM, and
wherein the second control signal is configured to cause the pump to operate at the specified RPM.

4. The FMS of claim 3, the memory further storing instructions, which when executed by the processing circuitry, cause the processing circuitry to:
receive, from the pump, an indication of a current RPM of the pump; and
determine the predicted ILP of the lumen at the time step in the future based in part on the current RPM of the pump and the specified RPM.

5. The FMS of claim 4, the memory further storing instructions, which when executed by the processing circuitry cause the processing circuitry to:
receive, from a medical device pressure sensor disposed in a distal end of the elongate shaft, an indication of a current ILP of the lumen; and
determine the predicted ILP of the lumen at the time step in the future based in part on the current ILP of the lumen, the current RPM of the pump, and the specified RPM.

6. The FMS of claim 5, the console comprising an FMS pressure sensor configured to measure a pressure in the fluid cassette, the memory further storing instructions, which when executed by the processing circuitry cause the processing circuitry to:
receive, from the FMS pressure sensor, an indication of a current pressure of the fluid cassette; and
determine the predicted ILP of the lumen at the time step in the future based in part on the current pressure of the fluid cassette, the current ILP of the lumen, the current RPM of the pump, and the specified RPM.

7. The FMS of claim 1, wherein the fluid pathway includes a fluid dampening chamber having a fluid inlet configured for fluid ingress into the fluid dampening chamber and a fluid outlet configured for fluid egress from the fluid dampening chamber, the memory further storing instructions, which when executed by the processing circuitry cause the processing circuitry to determine the predicted ILP of the lumen at the time step in the future based in part on fluid dynamics of the fluid dampening chamber.

8. A controller for a fluid management system (FMS), the FMS configured to provide fluid flow to an endoscopic medical device, the controller comprising:
a memory storage device comprising instructions; and
processing circuitry coupled to the memory and configured to:
receive an indication to operate a pump of the FMS at a specified parameter;
determine a predicted intraluminal pressure (ILP) of a lumen at a time step in the future, wherein the endoscopic medical device comprises an elongated shaft inserted into the lumen and is configured to provide fluid from the FMS to the lumen;
determine whether the predicted ILP is less than or equal to a threshold ILP level; and
send, responsive to a determination that the predicted ILP is not less than or equal to the threshold ILP level, a first control signal to the pump, the first control signal to cause the pump to operate at a parameter different than the specified parameter; or
send, responsive to a determination that the predicted ILP is less than or equal to the threshold ILP level, a second control signal to the pump, the second control signal to cause the pump to operate at the specified parameter.

9. The controller of claim 8, the memory storage device further storing instructions, which when executed by the processing circuitry cause the processing circuitry to:
solve a series of time-dependent differential equations; and
determine the predicted ILP of the lumen at the time step in the future based on the solution to the series of time-dependent differential equations.

10. The controller of claim 9, the memory storage device further storing instructions, which when executed by the processing circuitry cause the processing circuitry to solve the series of time-dependent differential equations using a forward-Euler algorithm or a backward-Euler algorithm.

11. The controller of claim 9, wherein the series of time-dependent differential equations relates a volume of the fluid cassette with the current ILP, relates fluid outflow resistance of the lumen to fluid inflow resistance of the lumen, and/or is based in part on compliance of the lumen.

12. A method for a controller of a fluid management system (FMS), the FMS configured to provide fluid flow to an endoscopic medical device, the method comprising:
receiving, at circuitry of the controller, an indication to operate a pump of the FMS at a specified parameter;
determining, by the circuitry, a predicted intraluminal pressure (ILP) of a lumen at a time step in the future, wherein the endoscopic medical device comprises an elongated shaft inserted into the lumen and is configured to provide fluid from the FMS to the lumen;
determining, by the circuitry, whether the predicted ILP is less than or equal to a threshold ILP level; and
sending, responsive to a determination that the predicted ILP is not less than or equal to the threshold ILP level, a first control signal to the pump from the circuitry, the first control signal to cause the pump to operate at a parameter different than the specified parameter; or
sending, responsive to a determination that the predicted ILP is less than or equal to the threshold ILP level, a second control signal to the pump from the circuitry, the second control signal to cause the pump to operate at the specified parameter.

13. The method of claim 12, wherein the specified parameter comprises a desired RPM and wherein the first control signal is configured to cause the pump to operate at an RPM different than the desired RPM.

14. The method of claim 12, wherein the first control signal is configured to cause the pump to stop or to reverse flow direction.

15. The method of claim 12, further comprising
receiving, at the circuitry, an indication to operate the pump at a specified RPM,
wherein the first control signal is configured to cause the pump to operate at an RPM less than the specified RPM, and
wherein the second control signal is configured to cause the pump to operate at the specified RPM.

16. The method of claim 15, wherein the FMS comprises a console configured to receive a fluid cassette, the pump, and an FMS pressure sensor configured to measure a pressure in the fluid cassette, and wherein the pump is configured to cause fluid to flow through the fluid cassette, the method further comprising:
receiving, at the circuitry from the pump, an indication of a current RPM of the pump;
receiving, at the circuitry from a medical device pressure sensor disposed in a distal end of the elongate shaft, an indication of a current ILP of the lumen;
receiving, at the circuitry from the FMS pressure sensor, an indication of a current pressure of the fluid cassette; and
determining, by the circuitry, the predicted ILP of the lumen at the time step in the future based in part on the current pressure of the fluid cassette, the current ILP of the lumen, the current RPM of the pump, and the specified RPM.

17. The method of claim 12, wherein the fluid cassette comprises a housing defining a fluid pathway therethrough, wherein the fluid pathway comprises a fluid dampening chamber having a fluid inlet configured for fluid ingress into the fluid dampening chamber and a fluid outlet configured for fluid egress from the fluid dampening chamber, the method further comprising determining, by the circuitry, the predicted ILP of the lumen at the time step in the future based in part on fluid dynamics of the fluid dampening chamber.

18. The method of claim 12, further comprising:
solving, by the circuitry, a series of time-dependent differential equations; and
determining, by the circuitry, the predicted ILP of the lumen at the time step in the future based on the solution to the series of time-dependent differential equations.

19. The method of claim 18, wherein the series of time-dependent differential equations relates a volume of the fluid cassette with the current ILP, relates fluid outflow resistance of the lumen to fluid inflow resistance of the lumen and/or is based in part on compliance of the lumen.

20. The method of claim 18, further comprising solving, by the circuitry, the series of time-dependent differential equations using a forward-Euler algorithm or a backward-Euler algorithm.
